(19) United States
(12) Patent Application Publication
(10) Pub. No.: US 2014/0324001 A1
(43) Pub. Date: Oct. 30, 2014

(54) CATHETER

(71) Applicant: Eric Serrano, Sharon, MA (US)

(72) Inventor: Eric Serrano, Sharon, MA (US)

(21) Appl. No.: 14/261,630

(22) Filed: Apr. 25, 2014

Related U.S. Application Data
(60) Provisional application No. 61/816,382, filed on Apr. 26, 2013.

(51) Int. Cl. A61M 25/00

(52) U.S. Cl. CPC A61M 25/0017; A61M 2205/18; A61M 2205/3368
USPC 604/328

(57) ABSTRACT

Provided in one embodiment is a system that includes a collection reservoir coupled to a flexible tube. The system may also include a temperature sensor disposed in the flexible tube and a control system. The control system may further include a processor configured to detect an amount of a fluid flowing in the flexible tube by detecting a temperature change in the flexible tube using the temperature sensor. The system may also include a signaling device. The signaling device may be activated by the processor responsive to the processor not detecting fluid flow in the flexible tube after a predetermined amount of time. In some embodiments, the system includes a full catheter system, and in some embodiments the system includes a coupling that may be added to off-the-shelf catheters.

### CROSS-REFERENCE TO RELATED APPLICATIONS

This application claims priority to U.S. Provisional Patent Application No. 61/816,382, filed on Apr. 26, 2013, which is incorporated herein by reference in its entirety.

### BACKGROUND

Catheter-induced infections account for a large percentage of healthcare-associated infections. It is estimated that catheter-induced infections cause 13,000 deaths annually. These preventable infections increase hospitals stays and may result in hundreds of millions of dollars of extra medical costs each year. These infections may often occur when hospital staff are unaware that a blockage has occurred within the catheter or when catheters remain in place for longer than necessary. To date no cost effective and easy method exists for detecting blockages within a urinary catheter.

### SUMMARY

In view of the foregoing, the Inventor has recognized and appreciated the advantages of systems and methods for detecting fluid flow through a catheter.

Accordingly, provided in one embodiment is a system that includes a collection reservoir coupled to a flexible tube. The system may also include a temperature sensor disposed in the flexible tube and a control system. The control system may further include a processor configured to detect an amount of a fluid in the flexible tube by detecting a temperature change in the flexible tube via the temperature sensor. The system may also include a signaling device. The signaling device may be activated by the processor responsive to the processor not detecting a fluid in the flexible tube after a predetermined amount of time.

In another embodiment, a system is provided for detecting an amount of a fluid flowing through a coupling. The system may include a coupling that at a distal end may be reversibly coupled to a drainage port of a catheter and at a proximal end may be reversibly coupled to a collection reservoir. The system may also include a temperature sensor disposed in the coupling and a control system. The control system may further include a processor configured to detect an amount of fluid flowing in the coupling by detecting a temperature change via the temperature sensor. The system may also include a signaling device. The signaling device may be activated by the processor responsive to the processor not detecting a fluid in the coupling after a predetermined amount of time.

In yet another embodiment, a method is provided for detecting an amount of fluid flowing through a catheter. The method may include detecting a first change in temperature in the catheter and correlating the first change in temperature to a first amount of a fluid flowing through the catheter. The method may also include detecting a second change in temperature and correlating the second change in temperature to a second amount of a fluid flowing through the catheter. The method may further include transmitting a notification when no second amount of the fluid flowing through the catheter is detected after the predetermined period of time.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIGS. 1A and 1B are block diagrams of a system configured to detect fluid flowing through a catheter according to two exemplary embodiments.

FIG. 2 is a block diagram of a coupling configured to detect fluid flowing through a catheter according to one exemplary embodiment.

FIG. 3 is a flow diagram of a method for detecting fluid flowing through a catheter according to one exemplary embodiment.

FIG. 4A is an image of a first prototype for detecting an amount of fluid flowing through a catheter according to one exemplary embodiment.

FIG. 4B is an image of a second prototype for detecting an amount of fluid flowing through a catheter according to one exemplary embodiment.

FIGS. 5A-5C are plots illustrating the change in resistance detected when a fluid flows through a catheter according to one exemplary embodiment.

### DETAILED DESCRIPTION

Following below are more detailed descriptions of various concepts related to, and embodiments of, inventive devices and methods for detecting flow through a catheter. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Urinary catheters may be a common source of healthcare-associated infections. These infections may increase morbidity rates, mortality rates, length of hospitalization, and ultimately healthcare costs. The system and methods described herein may increase patient safety and well-being. For example, they may facilitate the monitoring of a catheter, such as monitoring the fluid flow. While the examples described herein are primarily related to urinary catheters, one skilled in the art will easily recognize that the present disclosure need not be limited to urinary catheters. The methods and systems described herein may measure and detect flow through any type of medical catheter such as, but not limited to, catheters used for the administration of fluids to a patient, the collection of fluids from a patient, cardiac procedures, and hemodialysis.

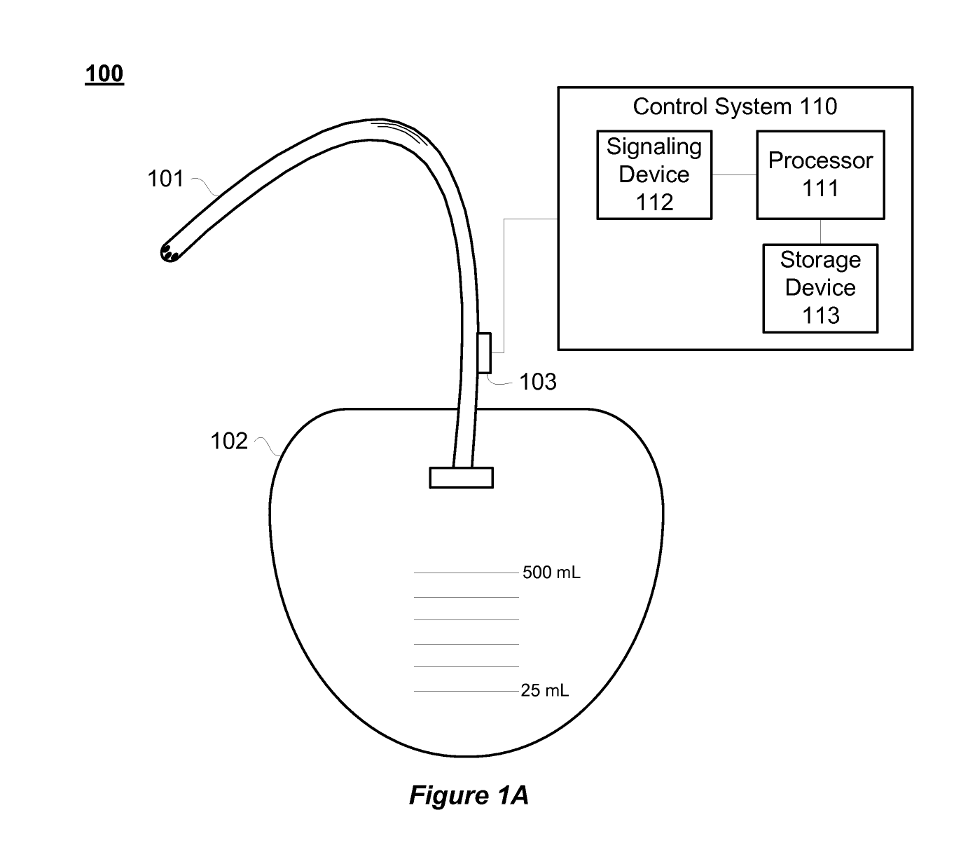

FIGS. 1A and 1B illustrate embodiments of a system 100 and 150, respectively, configured to monitor a fluid flow through a catheter. As a brief overview, the system 100 may include a flexible tube 101 coupled to a reservoir 102. A temperature sensor 103 may be disposed in or on the flexible tube 101. The temperature sensor 103 may communicate with a control system 110, and the control system 110 may include a processor 111, a signaling device 112, and a storage device 113.

Referring to FIG. 1A in greater detail, the flexible tube 101, as described above, may be any type of medical catheter. The catheter may be an urinary catheter. For example, the flexible tube 101 may be a Foley catheter. Other catheter examples may include Robinson catheters and external urinary catheters. The flexible tube 101 may include any suitable materials. For example, the material may include latex, polyurethane, nylon, thermoplastic elastomers, and/or silicone. The flexible tube 101 may have any cross-sectional geometry such as, but not limited to, a circular geometry. The flexible tube's geometry may have a diameter between about 1 mm and about 3 mm, about 3 mm and about 5 mm, about 5 mm and about 7 mm, and about 7 mm and about 10 mm. The flexible tube 101 may have any suitable length. For example, the length may be adjusted for the size and type of patient into which the flexible tube 101 is to be deployed. In some embodiments, the flexible tube 101 may include and/or be coated with an antimicrobial agent. The antimicrobial agent may be any suitable agent that may mitigate or prevent growth or attachments of microorganisms to the catheter, such as bacteria, viruses, etc. Similarly, the flexible tube 101 may include a surface coating that reduces friction when the flexible tube 101 is inserted into a patient. The end of the flexible tube 101 opposite the end coupled to the reservoir 102 may include one or more holes for the collection of fluid from the patient. In some embodiments, the tip of the flexible tube 101 including a plurality of holes may be shaped to a specific predetermined geometry and/or curved to facilitate easier passage through and insertion into a patient.

The flexible tube 101 may be coupled to a reservoir 102. The reservoir 102 may also be referred to as a drainage bag or collection bag. The reservoir 102 may be flexible, such as a drainage bag that may be worn by a patient discreetly under the patient's clothing. In other embodiments, the reservoir 102 may be rigid, such as a plastic collection cup. The reservoir 102 may be partially or wholly transparent such that fluid within the reservoir 102 may be observed. The transparent portions of the reservoir 102 may include markings (as shown in FIG. 1A) that provide an indication of the volume of fluid currently stored in the reservoir 102.

The system 100 may also include a temperature sensor 103. The temperature sensor 103 may be disposed in an internal lumen of the flexible tube 101 through which fluid flows from the patient to the reservoir 102. In some embodiments, the word “in” in the internal lumen with respect to the temperature sensor 103 may refer to temperature sensor 103's placement within the internal lumen of the flexible tube 101, within the material of the flexible tube 101, or the coupling of the temperature sensor 103 to the wall of the internal lumen. In one embodiment, the temperature sensor 103 may be coupled to the exterior wall of the flexible tube 101 such that the temperature sensor 103 detects temperature changes in the exterior wall of the flexible tube 101 that may be induced by the flowing of a fluid through the flexible tube 101. Specifically, the temperature sensor 103 may be employed to detect an amount of fluid flow through the flexible tube 101. In one embodiment, the detection is performed for detecting the fluid flow through the flexible tube 101 within a predetermined period of time. For example, in a case of the fluid including urine, as urine flows from a patient to the reservoir 102, the temperature sensor 103 may detect the heat radiated by the urine. It is noted that in this case the catheter may be referred to as a urinary catheter. In some embodiments, the temperature sensor 103 may include a thermocouple, thermistor, or similar device for detecting temperature changes. In other embodiments, the amount of fluid flow may be detected by a flow meter, such as an optical and/or mechanical flow meter. The temperature sensor 103 may be placed anywhere along the length of the flexible tube 101. In some embodiments, the temperature sensor 103 is placed proximate to the reservoir 102 to reduce the likelihood of detecting temperature changes that may be induced by a patient's own body temperature. In some embodiments, the reservoir 102 may be a component of a catheter collection bag. The catheter collection bag may be filled through a length of tubing. The length of tubing may be coupled to the reservoir 102 at one end and coupled to the flexible tube 101 at the opposite end. In some embodiments, the temperature sensor 103 may be disposed at any position along the collection bag's length of tubing, at any position along the flexible tube 101, and/or in the reservoir 102. The flexible tube 101 may be located at any desirable location. For example, the flexible tube 101 may be a component of the collection reservoir 102—e.g., of a collection bag.

Referring to FIG. 1B, system 150 illustrates an embodiment configured to monitor a fluid flow through a catheter. In some embodiments, the system 150 may include components similar to system 100. System 150 may also include a second temperature sensor 104. The temperature sensor 104 may have any of the characteristics and properties of the first temperature sensor 103 as described herein. In some embodiments, the temperature sensor 104 may be disposed near the tip of the catheter 101. For example, the temperature sensor 104 may be disposed in or on a point of the catheter 101 such that the temperature sensor 104 is disposed within a patient's bladder after placement of the catheter 101. In some embodiments, the temperature sensor 104 may measure the temperature within a patient's bladder. The temperature sensor 104 may be used to determine if the catheter 101 is properly placed within the patient. In other embodiments, data from the temperature sensor 104 may be used in conjunction with data from the temperature sensor 103 to detect the presence of a fluid flow and/or an amount of a fluid flowing through the catheter 101.

Referring again to FIGS. 1A and 1B, temperature changes detected by the temperature sensor 103 may be processed by a control system 110. In some embodiments, the control system 110 is coupled to the flexible tube 101 and/or reservoir 102. In other embodiments, the control system 110 may be housed separately from the flexible tube 101 and/or reservoir 102. For example, the control system 110 may comprise a patient monitoring device that is located bedside in a hospital environment. In another example, the control system 110 may be a part of a consumer electronic device, such as a portable consumer electronic device, portable display system, tablet computer, and/or portable reader. A consumer electronic device may include a smartphone, which may be worn and/or transported by the patient and/or hospital staff. In some embodiments, the above described flexible tube 101, reservoir 102, and temperature sensor 103 may be disposable and the control system 110 may be reusable. In other implementations, the control system 110 may also be disposable.

The control system 110 may include one or more processors 111 that execute machine-readable instructions. The instructions may be executed to perform any of the processes described herein. The instructions may be stored in a non-transitory computer readable medium. Such a medium may include CDs, DVDs, solid-state drives, flash memories, and/or hard-drives. In some embodiments, the processor 111 may be any logic circuit that responds to and processes machine-readable instructions. The processor may be a processing unit such as the processors manufactured by Microchip, Atmel, Intel, or any other single- or multi-core processor capable of operating as described herein.

As described above, the processor 111 may be instructed to detect temperature changes that may be indicative of an amount of fluid flowing through the flexible tube 101. The amount of fluid in some embodiments herein may refer to fluid flow. Depending on the context, “fluid flow” may refer to different aspects of a flowing fluid (e.g., the presence of a fluid flow, the amount of a fluid flow, the flow rate of a flowing fluid, and/or the pattern of a flowing fluid (e.g., laminar, turbulent, etc.)) The processor 111 may be configured such that it may discriminate between temperature changes that are indicative of flowing fluid and temperature changes that may not indicate a fluid flowing through the flexible tube 101. For example, the processor 111 may mark a first temperature change as caused by flowing fluid and mark a second temperature change as caused by the patient sitting on the temperature sensor 103. In some embodiments, the processor 111 may calculate the time between subsequent temperature changes indicative of flowing fluid through the flexible tube 101. For example, the processor 111 may activate the signaling device 112 when the processor determines that a fluid has not flowed through the flexible tube 101 within a predetermined amount of time.

In some embodiments, the processor 111 may detect and count the number of times a certain amount of fluid flows through the flexible tube 101 during a given time period. For example, the processor 111 may detect that a patient is urinating two times per hour. In another embodiment, the processor 111 may determine the length of time a fluid is flowing though the flexible tube 101. In some embodiments, the processor 111 may determine that urine flow rates of greater than 50 mL per hour are normal and flow rates below 50 mL per hour are abnormal. Responsive to detecting abnormal flow rates, the processor 111 may activate a signaling device 112, as described below. In other embodiments, the processor 111 may be configured to measure the length of time the catheter has been placed in the patient. For example, the catheter may start a timer when removed from its sterile packaging or when activated for the first time. The processor 111 may indicate, using the signaling device 112, the time accumulated on the timer, which may provide healthcare professionals with an estimate of the length of time the catheter has been in place. In some embodiments, the catheter may indicate to a healthcare professional when the catheter should be replaced.

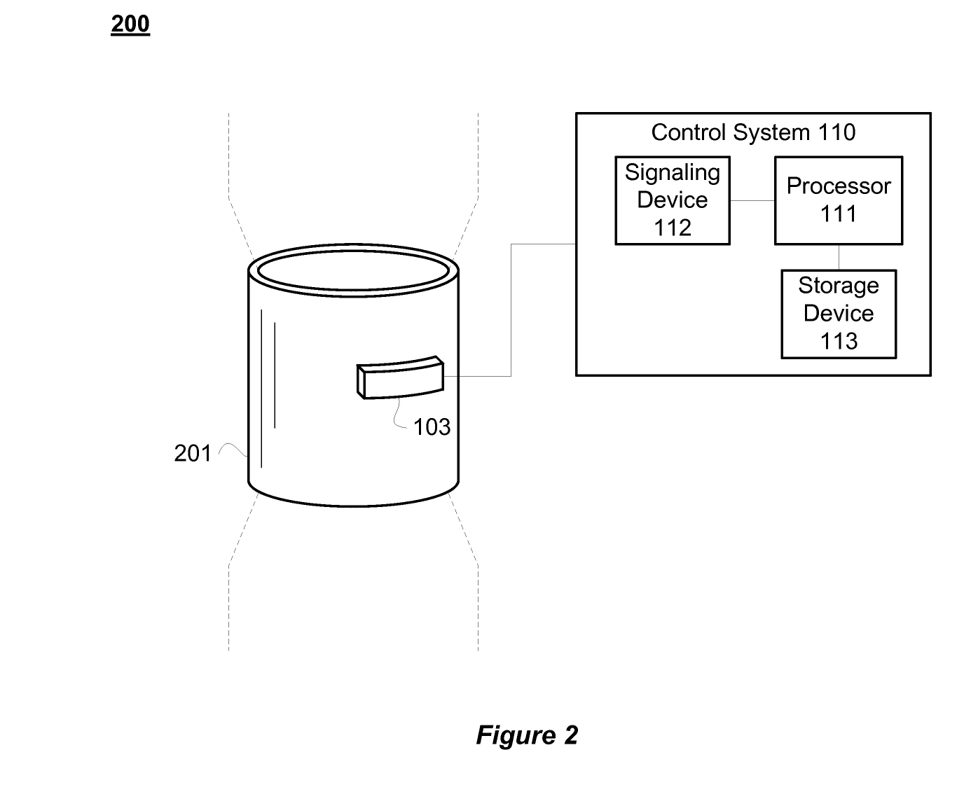

The signaling device 112 may alert a patient and/or medical staff to blocked flow in the flexible tube 101. A “blocked” flow may refer to a lack of fluid flow through the flexible tube 101, a reduced fluid flow through the flexible tube 101, and/or stagnant fluid (e.g., a fluid is present in the flexible tube 101 but is not flowing). For example, and as described in greater detail in relation to FIG. 3, the processor may determine that fluid is no longer flowing through the flexible tube 101 and alert medical caretakers using the signaling device 112 that the flexible tube 101 could possibly be occluded and/or positioned such that fluid may not flow to the reservoir 102 (e.g., the reservoir 102 may be placed at a height that does not allow fluid to be gravitationally forced to the reservoir 102). In other embodiments, the signaling device 112 may inform a patient and/or caretaker of the outcome of any of the above described processor 111 processes. The signaling device 112 may include, but is not limited to, a visual alarm and/or an auditory alarm. For example, the signaling device 112 may include one or more light emitting diodes (LED) that blink to convey information to medical professionals. For example, the LEDs may illuminate a first color (e.g., green) when the flexible tube 101 is not blocked, and illuminate a second color (e.g., red) when the flexible tube 101 is blocked. In another embodiment, the LEDs may blink at a specific rate that correlates to various processor 111 determinations. For example, the LEDs may blink at a first rate to indicate that a patient is urinating greater than a predetermined number of times per hour and may blink at a second rate when the patient is urinating less than the predetermined number of times per hour. In some embodiments, the predetermined amounts or levels described herein may be set by a medical professional and/or they may be preprogrammed into the instructions that are executed by the processor 111. Similarly, in embodiments where the signaling device 112 includes an auditory alarm, the signaling device 112 may generate tones at specific frequencies and/or durations to convey information similar to the above-described visual alarms. In some embodiments, the signaling device may include an LCD display. The processor may display information such as, but not limited to, at least one of the last time a patient urinated, the amount of time the catheter has been in place, and the frequency of the patient's urination.

In another embodiment, the signaling device 112 may include a wireless transmitter. When activated, the wireless transmitter may send a signal message to a receiving device. The receiving device may include a computer, smartphone, and/or patient monitor. The receiving device may contain therein a monitoring software program configured to execute monitoring process(es). For example, in a hospital setting, the transmitter may send a wireless alert to a computer at a nurses' station to alert a nurse that there may be a blockage in a patient's catheter. In some embodiments, the signal message may be transmitted responsive to the processor 111 not detecting a predetermined amount of fluid within a predetermined time period. In other embodiments, the signal message may be transmitted to the receiving device at a specific frequency. For example, the signal message may be a status message, which may be transmitted to a nurses' station every 15 minutes, 30 minutes, every hour, or at a longer interval. Shorter time intervals are also possible. The status message may include at least one of the current length of time the catheter has been in place, the last time since an amount of fluid was detected, and an indicator of a flagged status (e.g., the predetermined time between detections of fluid flows has been surpassed). The transmission of the signal message may be automated. The automation may be achieved by using at least one program configured to execute the flow detection and transmission processes.

The control system 110 may also include a storage device 113. In some embodiments, patient data generated by the system 100 may be stored in the storage device 113. For example, the storage device 113 may include a memory card or flash memory that may be removed from the control system 110 and downloaded into a general computing device (e.g., a laptop). In some embodiments, the storage device 113 may not be removable, but data may be written to and/or retrieved by methods such as, but not limited to, USB, Bluetooth, ad hoc wireless transmissions, and serial data protocols. In some embodiments, a user and/or medical professional may enter patient information into the system 100, which may be stored in the storage device 112. For example, a nurse may enter a patient's name into the system 100, which may then be displayed on an LCD of the signaling device 112. Additional examples of the storage device 113 may include hard drives, solid state hard drives, optical drives, EPROM, EEPROM or any other type of storage device.

While the embodiment of system 100 illustrates the control system 110 within a single housing, one of ordinary skill in the art may easily recognize that the components of the control system 110 do not need to be housed within a single unit. For example, the system 100 may transmit data to a program running on a computer separate from the system 100 that saves the patient data to the computer's hard drive.

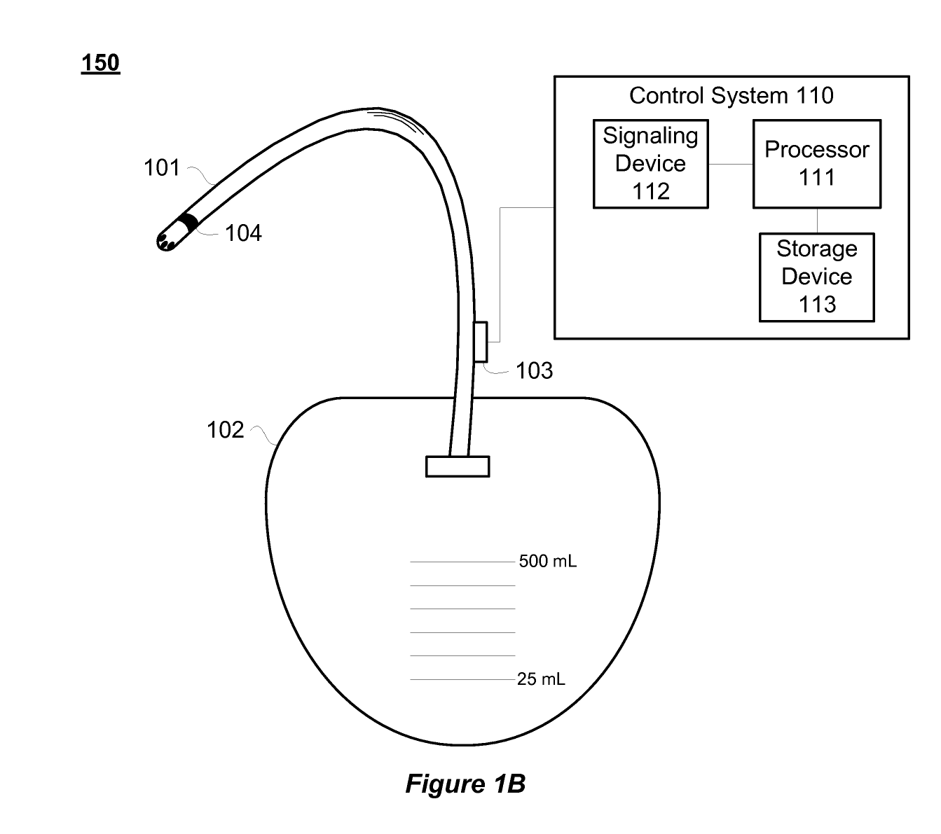

FIG. 2 provides an exemplary embodiment of system 200 for detecting fluid flow. Similarly to system 100, system 200 may include a temperature sensor 103 and control system 110 including a processor 111, a signaling device 112, and a storage device 113. The system 200 also includes a coupling 201. The temperature sensor 103 may be disposed in or on the coupling 201 such that it may detect fluid flow through the coupling 201. The coupling 201 may be configured such that it may be connected inline with any medical catheter and collection reservoir (represented by the dotted lines in FIG. 2). The term “inline” may refer to the coupling 201 being coupled to a medical catheter and/or a collection reservoir such that the coupling 201 does not obstruct and/or divert a substantial portion of a fluid as it flows from the medical catheter to the collection reservoir. For example, the coupling 201 may be part of a kit that may be added to an off-the-shelf catheter that does not contain the ability to detect fluid flow. The kit may be include a packaging material—e.g., a sterilization container, including, for example, sealable sterilization pouch or sterilization tray, the coupling 201, instructions for operating the device, and a disinfectant. Other materials suitable for a catheter kit may also be included. Thus, the system 200 may be added to catheters and similar devices to provide the functionality described above in relation to system 100.

FIG. 3 provides an exemplary method 300 for detecting fluid flow through a catheter. The method may include detecting a change in temperature (step 301) and correlating the change in temperature to fluid flowing through the catheter (step 302). The method 300 may also include detecting a second change in temperature (step 302) and correlating the second change in temperature to fluid flowing through the catheter (step 303). The method may also include transmitting a notification when no second change in temperature is detected (step 304).

As set forth above, the method 300 may begin with the detection of a first temperature change in a catheter (step 301). A temperature sensor 103 may be coupled to a flexible tube or catheter as described above. In one embodiment wherein the fluid is urine, as urine leaves the body at a temperature higher than typical ambient temperatures, the urine may be detected by a temperature change as the urine flows through the catheter. In some embodiments, the temperature sensor 103 may be disposed into the catheter such that the heat of the urine may be directly detected by the temperature sensor 103. In other embodiments, the temperature sensor 103 may be coupled to the catheter such that the temperature sensor 103 indirectly detects a temperature change. For example, the flowing fluid may transfer heat to the catheter, which may be detected by the temperature sensor 103. In some embodiments, the processor 111 may divide the signal received from the temperature sensor 103 into windows of a predefined length of time. The processor 111 may analyze each window to determine if fluid flowed through the catheter during the window. In some embodiments, the processor 111 may subdivide the windows into bins. The bins may be created when a plurality of samples from the temperature sensor 103 is averaged. For example, the system may set the window to 10 minutes. The processor 111 may then bin the window by sampling the temperature once every second for one minute and then average the samples to yield a single average temperature value for the minute long bin. The processor 111 may then determine if any of the averaged one-minute bins is above a predetermined threshold. In some embodiments, the predetermined threshold may be a specific temperature threshold (e.g., 90° F.); one, two, or three standard deviations above the temperature values of the previous bins; or a predetermined increase compared to the previous bin. When the processor 111 determines that one of the bins surpasses the predetermined threshold, the processor 111 may mark the window as containing a flow event.

The method 300 may continue by correlating the first change in temperature with an amount of fluid flowing through the catheter (step 302). In some embodiments, the temperature sensor 103 may detect temperature changes that are not indicative of a flowing fluid. In some embodiments, the processor 111 may use characteristics of the temperature change to determine if the detected temperature is indicative of a flowing fluid or caused by an artifact. For example, artifacts may include the patient sitting on the temperature sensor 103 or the temperature sensor 103's placement near a source of heat. Exemplary characteristics of the temperature change may include, but are not limited to, the percentage change in temperature, the maximum and/or minimum temperature during the temperature changing event, and/or the length time the temperature change persists. Furthering this example, a temperature change that lasts for several minutes may be indicative of a patient sitting on the temperature sensor and not of fluid flowing through the catheter. Responsive to determining that the first temperature change is induced by a flowing fluid and not a signaling artifact, the processor 111 may mark the time at which the temperature change was detected.

The method 300 may continue with the detection of a second temperature change (step 303). The second temperature change may be detected in a way similar to the first temperature change in step 301. In some embodiments, the system may enter a refractory period after detecting the first temperature change such that the second temperature change may not be detected until the system exits the refractory period. The “refractory period” may be a predetermined length of time during which the system may not analyze signals from the temperature sensor 103. The refractory period may ensure that a single flow event with a discontinuity, such as an air bubble or other gap in fluid flow, is not counted as a first and second temperature change.

The method 300 also includes correlating the second temperature change with an amount of fluid flowing through the catheter (step 304). The second temperature change may be correlated to a fluid flowing through the catheter in a method similar to step 302. The system may also mark the time at which the second temperature change occurred responsive to detecting the second temperature change. In some embodiments, the system may determine the length of time between the first and second temperature changes.

The method may further include the transmission of a notification when a second temperature change is not detected within a predetermined period of time (step 305). In some embodiments, the predetermined period of time may be between about 1 hr and about 2 hr, about 2 hr and about 3 hr, about 3 hr and about 4 hr, about 4 hr and about 5 hr, about 5 hr and about 6 hr, and about 6 hr to 24 hr. As described in relation to FIGS. 1A-1B and 2, the transmission of the notification may include generating a signal with the signaling device 112. In some embodiments, the notification may be transmitted to a monitor station—e.g., a patient monitor, a nurse's station, and/or a program executing on a computer or smartphone. The monitor may be attached or separated from the catheter.

### NON-LIMITING WORKING EXAMPLES

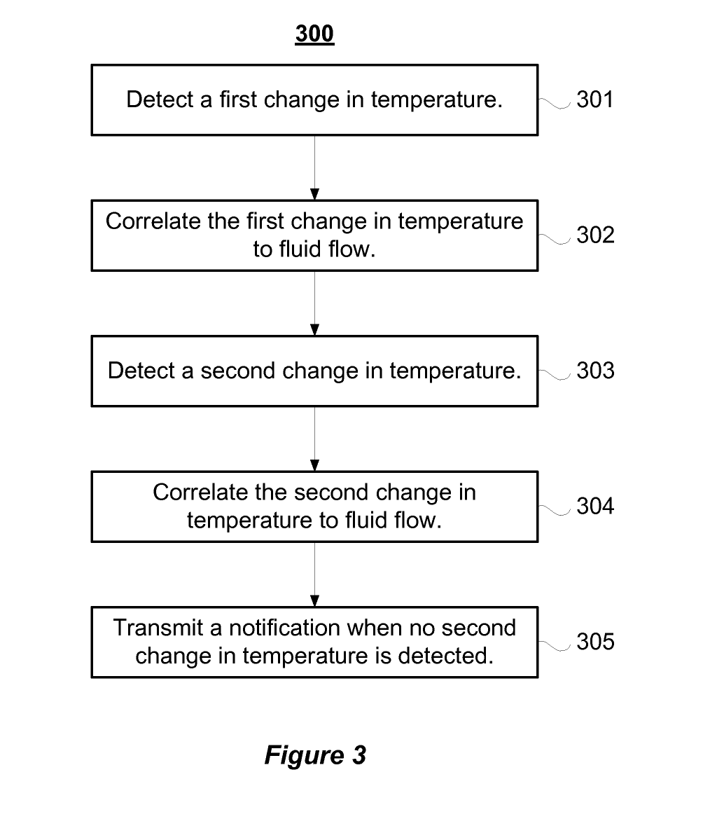

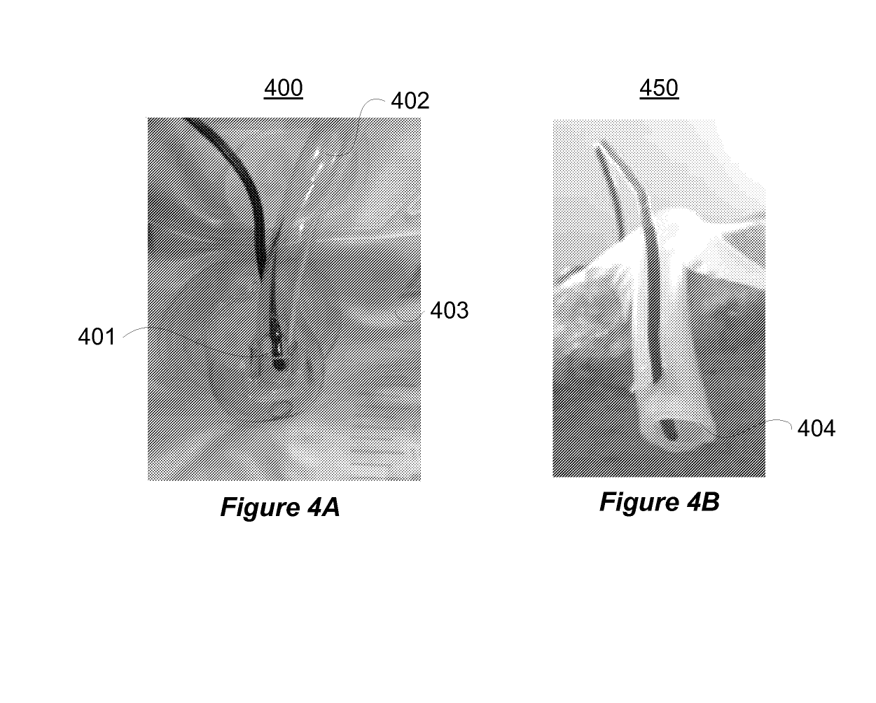

FIGS. 4A and 4B illustrate exemplary prototype embodiments according to the embodiments described herein. In the prototype 400, a temperature sensor 401 was disposed near the coupling between a flexible tube 402 and a collection reservoir 403. In this embodiment, the flexible tube 402 was a component of the collection reservoir 403. In this embodiment, a distal end of the flexible tube (not pictured) was configured to reversibly couple to a proximal end of a catheter 404. As illustrated, the temperature sensor 401 was disposed within the interior lumen of the flexible tube 402; however, as described above, the temperature sensor 401 is not limited to the placement illustrated in FIG. 4A (e.g., the temperature may be coupled to an exterior or interior wall, or placed within the wall of the flexible tube 402). In the prototype 450, the temperature sensor 401 was disposed within an interior lumen of a catheter 404. In some embodiments, the prototypes 400 and 450 may be used together or independently. For example, the processor 111 may need a detected temperature change at a temperature sensor disposed within the catheter and a temperature sensor disposed within the collection reservoir before associating a temperature change with an amount of fluid flow.

Referring again to FIGS. 4A and 4B, the prototypes 400 and 450 detected a fluid flow through the prototype by sampling from the temperature sensor 401 every second. The temperature sensor 401 was a thermistor (i.e., a resistor whose resistance varies with temperature) in the prototypes 400 and 450. In the prototypes 400 and 450, a constant voltage was applied across the thermistor and a resistor of known resistance. Every second the processor (not shown in FIGS. 4A and 4B) measured the voltage at a point between the first and second resistors and, using Ohm's law, calculated the present resistance of the thermistor. The processor averaged the resistance readings from each minute to generate an average resistance for the given minute (or bin as referenced above). The prototypes 400 and 450 stored ten one-minute bins to create a 10 minute window. The processor determined that an amount of fluid flowed through the prototype if a bin (where n is the current bin) had a resistance 98.3% less than its previous bin (n−1). In memory, the processor would flag the window as having or not having a flow state (i.e., detecting or not detecting an amount of fluid flow through the prototype). The above process would then repeat for each subsequent time window.

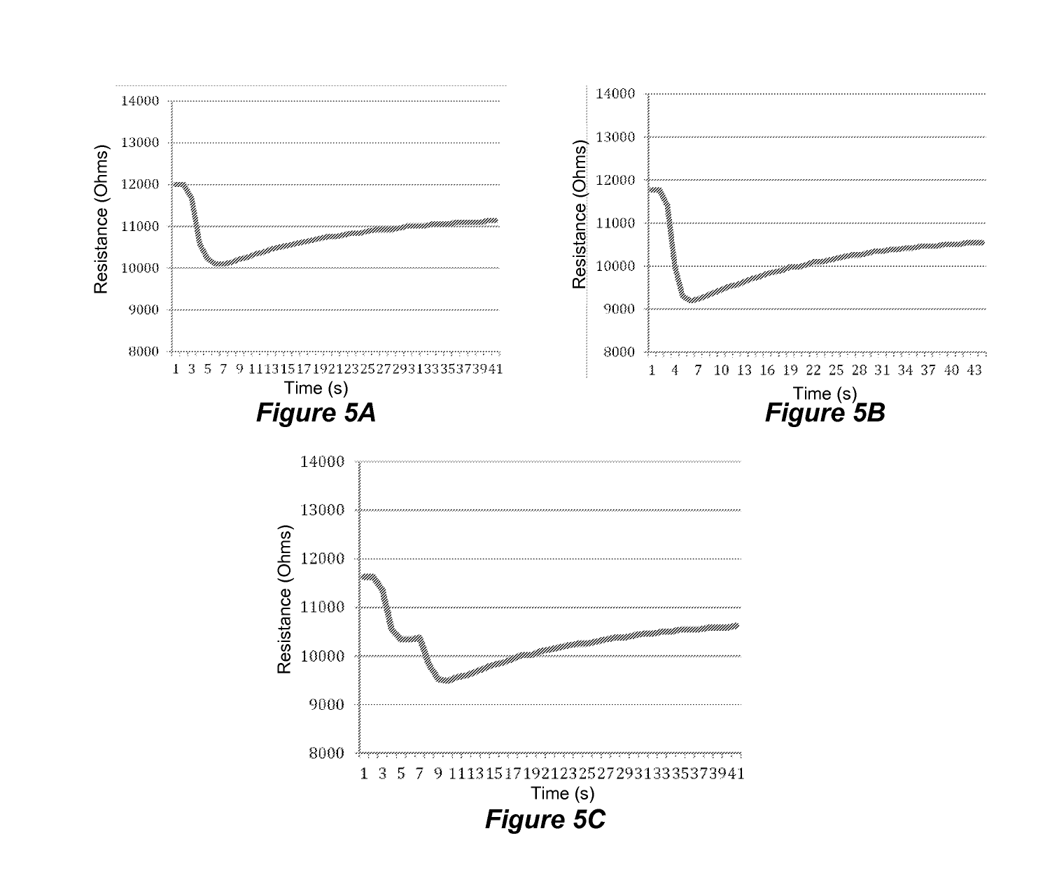

FIGS. 5A-5C are plots illustrating the changes in resistance recorded by prototypes described above in relation to FIGS. 4A and 4B in one example. During various experiments, 100° F. water was flowed through the prototype. As the water flowed through the prototype, the temperature sensor measured the change in temperature (as a change in resistance) that occurred within the catheter. FIG. 5A illustrates an experiment when approximately 10 mL of water was flowed through the catheter. In FIG. 5B, approximately 20 mL of water was flowed through the catheter, and in FIG. 5C two 5 mL boluses were consecutively flowed through the catheter several seconds apart. As illustrated in FIGS. 5A and 5B, the device was able to detect a flowing fluid and also detected a greater change in resistance for greater amounts of fluid flowing through the device. FIG. 5C illustrates that the prototype was able to detect boluses that occurred in rapid sequence.

### CONCLUSION

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments of the invention may be implemented in any of numerous ways. For example, some embodiments may be implemented using hardware, software or a combination thereof. When any aspect of an embodiment is implemented at least in part in software, the software code may be executed on any suitable processor or collection of processors, whether provided in a single computer or distributed among multiple computers.

In this respect, various aspects of the invention may be embodied at least in part as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium or non-transitory medium) encoded with one or more programs that, when executed on one or more computers or other processors, perform methods that implement the various embodiments of the technology discussed above. The computer readable medium or media may be transportable, such that the program or programs stored thereon may be loaded onto one or more different computers or other processors to implement various aspects of the present technology as discussed above.

The terms “program” or “software” are used herein in a generic sense to refer to any type of computer code or set of computer-executable instructions that may be employed to program a computer or other processor to implement various aspects of the present technology as discussed above. Additionally, it should be appreciated that according to one aspect of this embodiment, one or more computer programs that when executed perform methods of the present technology need not reside on a single computer or processor, but may be distributed in a modular fashion amongst a number of different computers or processors to implement various aspects of the present technology.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, the technology described herein may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” Any ranges cited herein are inclusive.

The terms “substantially” and “about” used throughout this Specification are used to describe and account for small fluctuations. For example, they may refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed:

1. A device comprising:
a collection reservoir coupled to a first end of a flexible tube;
a temperature sensor disposed in the flexible tube;
a control system, wherein the control system further comprises:
a processor configured to detect an amount of a fluid in a flexible tube by detecting a temperature change using the temperature sensor; and
a signaling device, wherein the signaling device is activated by the processor responsive to the amount of the fluid in the flexible tube after a predetermined amount of time

2. The device of claim 1, wherein the flexible tube is a urinary catheter.

3. The device of claim 1, wherein the flexible tube is a component of a collection reservoir.

4. The device of claim 1, wherein the fluid comprises urine.

5. The device of claim 1, wherein the signaling device is configured to generate at least one of an auditory alert and a visual alert.

6. The device of claim 1, wherein the signaling device further comprises a transmitter.

7. The device of claim 1, wherein the signaling device further comprises a transmitter configured to wirelessly transmit an alert to a monitoring station.

8. The device of claim 1, further comprising a display configured to display a length of time since a last detection of the fluid flowing through the flexible tube.

9. A device comprising:
a coupling, wherein a distal end of the coupling is configured to reversibly couple to a drainage port of a catheter and a proximal end of the coupling is configured to reversibly couple to a collection reservoir, and wherein a fluid flows through a lumen of the coupling from the catheter to the collection reservoir;
a temperature sensor disposed in the lumen of the coupling;
a processor configured to detect an amount of the fluid flowing through the coupling by measuring a temperature change; and
a signaling device that is activated by the processor responsive to the amount of the fluid flowing through the lumen of the coupling in a predetermined amount of time.

10. The device of claim 9, wherein the signaling device is configured to generate at least one of an auditory alert and a visual alert.

11. The device of claim 9, wherein the signaling device further comprises a transmitter.

12. The device of claim 9, wherein the signaling device further comprises a transmitter configured to wirelessly transmit an alert to a monitoring station.

13. The device of claim 9, further comprising a display configured to display a length of time since a last detection of the fluid flowing through the lumen of the coupling.

14. The device of claim 9, wherein the fluid comprises urine.

15. A method comprising:
detecting a first change in temperature in a catheter;
correlating the first change in temperature to a first amount of a fluid flowing through the catheter;
detecting a second change in temperature in the catheter after a predetermined period of time;
correlating the second change in temperature to a second amount of a fluid flowing through the catheter; and
transmitting a notification when no second amount of a fluid flowing through the catheter is detected after the predetermined period of time.

16. The method of claim 15, wherein transmitting further comprises wirelessly transmitting the notification to a monitoring system.

17. The method of claim 15, wherein transmitting further comprises generating one of an auditory alter and a visual alert.

18. The method of claim 15, further comprising detecting a total number of temperature changes over a second predetermined period of time.

19. The method of claim 15, further comprising transmitting a notification when a total number of temperature changes over a second predetermined period of time is below a predetermined threshold.

20. The method of claim 15, further comprising detecting a time duration of the first change in temperature and detecting a time duration of the second change in temperature.

21. The method of claim 15, further comprising transmitting a notification when one of a time duration of the first change in temperature and a time duration of the second change in temperature is below a predetermined threshold.
